SEQUENCE LISTING (8 sequences)

SEQ ID NO: 1 - DNA, 21 bp, Artificial Sequence
  HSTYRO1E
nttntntntttttnntttttn

SEQ ID NO: 2 - DNA, 21 bp, Artificial Sequence
  HSTYRO1E
tnnnntntntntnnntntnnn

SEQ ID NO: 3 - DNA, 21 bp, Artificial Sequence
  HSTYRO1E
ntntntntnnntntntntntn

SEQ ID NO: 4 - DNA, 23 bp, Artificial Sequence
  HSU03039
ntttntttntntntntnttttnt

SEQ ID NO: 5 - DNA, 21 bp, Artificial Sequence
  HSU03039
tntntntnntntttnntntnt

SEQ ID NO: 6 - DNA, 21 bp, Artificial Sequence
  Inverted SEQ ID No.1 control
ntttttnntttttntntnttn

SEQ ID NO: 7 - DNA, 21 bp, Artificial Sequence
  Scrambled SEQ ID No. 1 control
ttnttnttnttnttnttnttn

SEQ ID NO: 8 - DNA, 2384 bp, Homo sapiens
  misc_feature (1)..(2384): Human tyrosinase gene - NCBI accession number M27160
tattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaagtcagtcatgtgcttttcagaggatgaaagcttaagataaagactaaaagtgtttgatgctggaggtgggagtggtattatataggtctcagccaagacatgtgataatcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

(19) United States
(12) Patent Application Publication
(10) Pub. No.: US 2010/0047192 A1
(43) Pub. Date: Feb. 25, 2010

(54) Novel tyrosinase-specific anti-gene oligonucleotides as depigmenting agents

(75) Inventors: Robin Kurfürst, Olivet (FR); Mathilde Bonnet-Duquennoy, Vic Le Comte (FR); Kristell Lazou, Orleans (FR); Carine Giovannangeli, Paris (FR)

Correspondence Address: HARNESS, DICKEY & PIERCE, P.L.C., P.O. BOX 828, BLOOMFIELD HILLS, MI, 48303, US

(73) Assignees: L.V.M.H. RECHERCHE, SAINT JEAN DE BRAYE (FR); CENTRE NATIONAL DE LA RECHERCHE SCIENTIFIQUE (CNRS), PARIS (FR); MUSEUM NATIONAL D'HISTOIRE NATURELLE, PARIS (FR)

(21) Appl. No.: 11/991,317

(22) PCT Filed: Sep. 1, 2006
(86) PCT No.: PCT/EP2006/065902

§ 371 Date: Feb. 29, 2008

(30) Foreign Application Priority Data
Sep. 1, 2005 (FR) 0508981

(51) Int. Cl. A61K 8/60; C07H 21/00; A61K 31/7088; A61Q 17/04; A61Q 19/02

(52) U.S. Cl. 424/59; 536/23.1; 514/44 R; 424/62

(57) ABSTRACT

The present invention relates to an anti-gene oligonucleotide which hybridizes specifically with the gene encoding human tyrosinase by Hoogsteen pairing between the complementary bases, said oligonucleotide forming a triple helix structure with the human tyrosinase gene. The invention also relates to the use of said oligonucleotide as a skin-depigmenting or skin-bleaching agent in a cosmetic composition or in a dermatological composition.

The present invention relates to an anti-gene oligonucleotide which hybridizes specifically with the gene encoding human tyrosinase by Hoogsteen pairing between the complementary bases, said oligonucleotide forming a triple helix structure with the human tyrosinase gene. The invention also relates to the use of said oligonucleotide as a skin-depigmenting or—bleaching agent in a cosmetic composition or in a dermatological composition.

In humans, pigmentation results from the synthesis and distribution of melanin pigments in the skin, the hair follicles or the eyes. Pigmentation is genetically predefined, but it is regulated by numerous internal or external factors. The melanins produced by melanocytes and also the number of melanocytes, their tyrosinase activity and their ability to export melanins to keratinocytes, and the size of the melanosomes which contain melanin granules, will condition the colour of human skin. For each individual, the colour of the skin varies mainly according to how much or how little irradiation it receives from ultraviolet (UV) rays. In other words, for each individual, there is a basic skin pigmentation when said individual is subjected to the weakest UV irradiation, corresponding to his or her lightest skin colour, and a more intense skin pigmentation if he or she receives a stronger UV irradiation, ranging up to the maximum pigmentation corresponding to his or her darkest skin colour when subjected to sustained exposure to intense UV irradiation.

Furthermore, as is well known, a very large genetic diversity with regard to skin pigmentation exists in the worldwide population. Thus, according to populations, the skin colour corresponding to the basic pigmentation defined above has a darker or lighter shade which lies between the two extremes: very light and very dark. Also according to populations, the difference in skin shade between the basic pigmentation and the maximum pigmentation is more or less great. Thus, it is well known that individuals belonging to certain populations with light skin (basic pigmentation) react rapidly and/or considerably to the action of UV radiation and can therefore readily exhibit skin with a dark shade, even when these individuals have not intentionally exposed themselves to the sun for a prolonged period. In the subsequent text, these individuals will be denoted by the expression “individuals very reactive to UV radiation”. This is in particular true of populations of Asian origin or of certain “mixed race” populations.

Moreover, individuals experience the appearance on their skin, in particular on the face or the hands, of areas and/or spots which are darker and/or more coloured, conferring a colour heterogeneity on the skin. These spots are due to a large concentration of melanin in the epidermal keratinocytes resulting from an exacerbated melanogenic activity in the melanocytes.

The mechanism of formation of skin pigmentation involves the synthesis of melanins. This mechanism is particularly complex and involves, schematically, the following main steps:

Tyrosine→Dopa→Dopaquinone→Dopachrome→Melanins

Tyrosinase and tyrosinase-related-protein 1 (TRP-1) are essential enzymes involved in this series of reactions. They catalyse in particular the reaction for conversion of tyrosine to Dopa (dihydroxyphenylalanine) and the reaction for conversion of Dopa to Dopaquinone, resulting in the formation of melanin pigments.

A molecule is recognized to be a depigmenting molecule if it acts directly on the epidermal melanocytes by inhibiting the activity of these cells and/or if it blocks one of the steps of melanin biosynthesis or else if it degrades the melanin formed. This is in particular the case when the molecule inhibits one of the enzymes involved in melanogenesis or when it reacts with the chemical compounds of the melanin synthesis chain.

Known depigmenting substances are in particular hydroquinone and derivatives thereof, ascorbic acid and derivatives thereof, placental extracts, kojic acid, arbutin, iminophenols, the combination of carnitine and quinone, aminophenol amide derivatives, and benzothiazole derivatives. These substances can have certain drawbacks. They can be unstable, can require use at high concentrations, can lack specificity with regard to their method of action, or can have a cytotoxic or irritant capacity.

The topical use of depigmenting substances which are effective and harmless is particularly sought in cosmetics and in dermatology. These substances are in particular used for treating regional hyperpigmentations due to melanocyte hyperactivity, such as idiopathic melasmas, localized hyperpigmentations due to melanocyte hyperactivity, such as pigmentary spots referred to as solar lentigo and senile lentigo, accidental hyperpigmentations such as photosensitization or post-lesional cicatrization, and also certain forms of leucoderma such as vitiligo. In the latter cases, if it is not possible to repigment the skin, the pigmentation at the periphery of the depigmented zones is reduced so as to give the skin a more homogeneous colour.

Depigmenting substances are also used as skin-bleaching agents by certain individuals, in particular those denoted above, who are very reactive to UV radiation, so as to lighten their colouring, in particular that of their face and their hands, in order to maintain a skin colour which is as light as possible or at the very least to reduce the pigmenting effects of UV rays.

The problem with which professionals are confronted is therefore the design, production or isolation of novel depigmenting substances or of novel bleaching agents for human skin and/or for body hair, which do not have the drawbacks of the known substances, i.e. which are non-irritant, non-toxic and/or non-allergenic for the skin and are stable in a composition.

The use of antisense oligonucleotides for treating a melanocyte dysfunction has been described in application US 20040014700. Moreover, certain strategies for obtaining depigmentation consist in blocking tyrosinase synthesis by using a double-stranded RNA oligonucleotide (FR 2840217). In the above applications, the biological target under consideration is the messenger RNA.

The object of the present invention consists in providing a depigmenting agent which acts on the melanogenesis process, and which is intended, firstly, in the case of a substantially homogeneous pigmentation, for bleaching the skin and/or body hair, i.e. decreasing the pigmentation thereof, and, secondly, for combating skin hyperpigmentation, i.e. when the skin exhibits pigmentation heterogeneity.

The inventors of the present invention have found that oligonucleotides which can hybridize with the gene encoding tyrosinase and only with this gene have a depigmenting activity.

Contrary to the mRNA-targeting strategy described above, that of the present invention therefore consists in using an oligonucleotide capable of hybridizing with the gene itself. An advantage of this is that the oligonucleotides of the present invention can hybridize with the gene in the form of a DNA double strand, whereas the oligonucleotides described in particular in applications US 20040014700 or FR 2840217 do not have this capacity. Another advantage of this novel strategy is that the gene is an entity which is less abundant than the messenger RNA or the protein for which it encodes, since most genes have two alleles in diploid cells. As a result, this activity exists even at a very low concentration, which increases the advantage of these oligonucleotides. Furthermore, the oligonucleotides according to the invention do not exhibit any cytotoxicity and can be synthesized, characterized, and isolated with a high degree of purity, in an industrial manner.

The oligonucleotides according to the invention therefore intervene considerably upstream of the melanogenesis mechanisms, by interacting directly on the tyrosinase gene so as to modulate the expression thereof and, consequently, inhibit the expression of the messenger RNA encoding tyrosinase. This results in a decrease in the amount of tyrosinase in the melanocytes. The oligonucleotides according to the invention offer an ideal solution to the problems posed by the substances conventionally used. The known substances which inhibit tyrosinase activity have multiple unacceptable side effects due to their weak specificity. Among the various sequences tested, we have found that the sequence SEQ ID No. 1 described hereinafter solves the problems encountered by the prior researchers by modulating as far upstream as possible the expression of the tyrosinase gene and, consequently, the production of the messenger RNA encoding tyrosinase, and therefore that of this enzyme, instead of directly inhibiting tyrosinase so as to obtain the depigmenting effect.

### DESCRIPTION

Thus, in a first aspect, the invention relates to an anti-gene oligonucleotide comprising a sequence of 15 to 25 nucleotides comprising the sequence 5′-C*TTC*TC*TC*TTTTTC*C*TTTTTC*-3′ (SEQ ID No. 1, C* denoting a 5-methylcytosine) which hybridizes specifically with the gene encoding human tyrosinase by Hoogsteen pairing between the complementary bases, said oligonucleotide forming a triple helix structure with the human tyrosinase gene. Preferably, this anti-gene oligonucleotide comprises between 18 and 21 nucleotides or between 21 and 25 nucleotides, preferably 21 nucleotides.

In the context of the present invention, the expression “gene encoding tyrosinase” is intended to mean the genomic sequence of the tyrosinase gene.

The oligonucleotides according to the invention hybridize directly to the gene formed from a DNA double helix in the cell. They thus make it possible to realize a final modulation of the amount of messenger RNA encoding tyrosinase and therefore of this enzyme produced by the gene.

Unlike the inventions described in documents US 20040014700 and FR 2840217, which are based on oligonucleotides forming hydrogen bonds according to the Watson-Crick pairing scheme so as to form a double helix structure, in the present document, the term “hybridization” is used to denote the formation of hydrogen bonds, also known as Hoogsteen or reserve-Hoogsteen pairing, between, firstly, the complementary bases, usually distributed on two strands of nucleic acid forming a double helix according to the Watson-Crick scheme, and, secondly, a third strand so as to form a triplex, a triple helix structure.

The degree of complementarity between the nucleic acid sequences is determined by comparing, after alignment, the first sequence with the sequence complementary to the second sequence. The degree of complementarity is calculated by determining the number of complementary positions for which the nucleotide is complementary between the two sequences thus compared, dividing this number of complementary positions by the total number of positions and multiplying the result obtained by 100 so as to obtain the degree of complementarity between these two sequences, expressed as a percentage.

The term “specific hybridization” means in particular that there is a sufficient degree of complementarity to avoid the nonspecific binding of the oligonucleotide to a non-targeted sequence under the conditions where specific binding is desired. The oligonucleotides according to the invention hybridize, preferably specifically, with the gene encoding tyrosinase. In particular, the oligonucleotides of the invention are capable of hybridizing only with the DNA of the genes which encode tyrosinase. The oligonucleotides according to the invention comprise a number of nucleotides which is sufficient in terms of identity and number to hybridize specifically. A subject of the present invention is thus oligonucleotides which hybridize specifically upstream of and/or with the region having the gene translation initiation codon.

In the context of the present invention, the expression “DNA which encodes tyrosinase” is intended to mean both the exons and the introns, in particular the exons.

A subject of the present invention is also oligonucleotides comprising one or more chemical modifications in their sugar components, their nucleobase components or their internucleotide backbone, which confer desirable physicochemical characteristics on the oligonucleotides according to the invention, such as an increased bioavailability, an increase in affinity for the target sequences, an increase in cellular internalization or a better biological stability, or an increase in stability in the presence of cellular nucleases.

By way of example, the modifications which can confer these characteristics are Locked Nucleic Acids (LNAs), Peptide Nucleic Acids (PNAs), 2′-O-alkyl derivatives and 2′-O-fluoro derivatives on the sugar component of the nucleoside, and phosphorothioate derivatives or methylphosphonate derivatives on the internucleotide backbone, or else derivatives bearing, in the 5′-position and/or 3′-position, a reactive group of the intercalating type such as acridine or which make it possible to establish a bridge with the target sequence, such as psoralene or an azide group.

The term “PNA” is known to the those skilled in the art and signifies a chemical structure similar to that of DNA but in which the backbone is composed of repeat units of N-(2-aminoethyl)glycine connected by peptide bonds. The various purine and pyrimidine bases are linked to the backbone by methylenecarbonyl bonds. The structure is presented below:

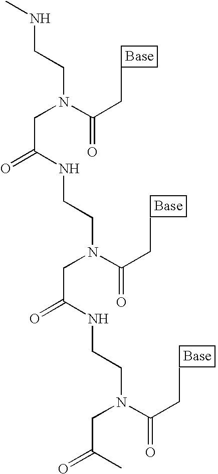

The term “LNA” is known to those skilled in the art and denotes a nucleic acid analogue containing a 2′-O,4′-C methylene bridge. This bridge blocks the flexibility of the ribofuranose ring by forming a rigid bicyclic structure. The structure is presented below:

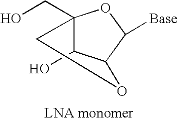

In the present document, the term “oligonucleotide” refers to polynucleotides formed from natural nucleobases and from pentafuranosyl (sugar) groups forming nucleosides which are connected to one another by native phosphodiester bonds. The term “oligonucleotides” therefore refers to the natural species or to the synthetic species formed from natural subunits or from close homologues thereof.

The term “oligonucleotides” can also refer to components which have similar functions to the natural oligonucleotides but which can have non-natural portions. The oligonucleotides can have sugar components, nucleobase components or internucleotide bonds which have been modified. As indicated above, among the possible modifications, the preferred modifications are Locked Nucleic Acids (LNAs) as described by Braasch DA and Corey DR (Chem. Biol. 2001, 8, 1-7), N3′-P5′ phosphoramidate derivatives as described by Faria M and Giovannangeli C. (J. Gene Med 2001, 3, 299-310), Peptide Nucleic Acids (PNAs) described by Wang G and Xu XS (Cell Res. 2004, 14, 111-118), 2′-O-alkyl derivatives on the sugar component, in particular 2′-O-ethyloxymethyl or 2′-O-methyl derivatives, and/or phosphorothioates or methylphosphonates for the internucleotide backbone or else derivatives bearing, in the 5′-position and/or 3′-position, a reactive group of the intercalating type such as acridine, or which make it possible to establish a bridge with the target sequence, such as psoralene or an azide group.

Chimeric oligonucleotides are included in the preferred modifications of the invention. The oligonucleotides contain at least two regions which are chemically different, each comprising at least one nucleotide. This involves in particular one or more regions comprising a modified nucleotide which confers one or more beneficial properties, such as, for example, a better biological stability, an increased bioavailability, an increase in cellular internalization or an increase in affinity for the target DNA.

Preferably, the chimeric oligonucleotides according to the invention are locked nucleic acids (LNAs), N3′-P5′ phosphoramidate derivatives, peptide nucleic acids or molecules with an internucleotide backbone which can be, entirely or partly, phosphodiesters, phosphorothioates or methylphosphonates, or combinations of phosphodiester and/or phosphorothioate and/or methylphosphonate bonds.

The term “oligonucleotides” can also refer to oligonucleotides onto which has been grafted a circular administration vector of the plasmid type or a linear administration vector of the nucleic acid or peptide type.

A subject of the present invention is also a cosmetic or dermatological composition containing at least one oligonucleotide described above and at least one cosmetically or dermatologically acceptable excipient or carrier. Such a composition can also contain one or more active ingredients. This or these active ingredient(s) is/are optionally aimed at complementing or strengthening the desired depigmenting effects.

The present invention also relates to the use of oligonucleotides capable of hybridizing specifically with the genes encoding tyrosinase, as cosmetic agents, in particular for depigmenting and/or bleaching the skin or body hair, and/or for reducing pigmentary spots on human skin.

In particular, the present invention comprises cosmetic compositions using these oligonucleotides as cosmetic agents. They are intended in particular to lighten skin colouring, to prevent or treat the formation of pigmentary spots due to the action of radiation from the sun, to reduce pigmentary senescence spots (senile lentigo), in particular on the hands, or else to lighten the hair on the body or the limbs.

The present invention also relates to the use of the oligonucleotides described above for the manufacture of a dermatological medicament for use in the treatment or prevention of diseases reflected by the overexpression and the overactivity of tyrosinase, by topical administration. This medicament may be for use in treating or preventing regional hyperpigmentations due to melanocyte hyperactivity, such as idiopathic melasmas, accidental hyperpigmentations such as photo-sensitization or in certain cases of post-lesional cicatrization, and for reducing pigmentary contrasts in certain forms of leucoderma such as vitiligo.

A subject of the present invention is also a depigmenting composition characterized in that it contains, in a cosmetically and/or dermatologically acceptable medium, an oligonucleotide sequence directed against the gene encoding tyrosinase.

The cosmetic composition and the medicament according to the invention are suitable for topical use and therefore contain a cosmetically or dermatologically acceptable medium, i.e. a medium compatible with the skin. The oligonucleotide sequence according to the invention may be present in an amount ranging from 0.00001% to 10%, and preferably from 0.0003% to 3%, of the total weight of these compositions.

The compositions of the invention can be in any of the forms suitable for topical application, in particular in the form of an aqueous, aqueous-alcoholic or oily solution, a simple or multiple, oil-in-water or water-in-oil emulsion, an aqueous or oily gel, a liquid, pasty or solid anhydrous product, an aqueous or oily dispersion of solid particles, such as polymeric nanospheres or nanocapsules, or else an aqueous dispersion of lipid vesicles of ionic or non-ionic type, as described in French patent FR 2534487.

These compositions may be more or less fluid and may have the appearance of a white or coloured cream, an ointment, a milk, a lotion, a serum, a paste or a foam. They can optionally be applied to the skin in the form of an aerosol. They can also be in pulverulent or non-pulverulent solid form, for example in the form of a stick. They can also be in the form of single doses, of patches, of pencils or of applicators which allow localized application on the spots of the face or the hands.

The cosmetic composition can in particular be formulated so as to be used as a care product and/or as a make-up product.

In a known manner, the compositions of the invention can also contain the adjuvants which are usual in the cosmetics and dermatological fields, such as hydrophilic or lipophilic gelling agents, hydrophilic or lipophilic active agents, preserving agents, antioxidants, solvents, fragrances, fillers, sunscreens, pigments, odour absorbers and dyestuffs. The amounts of these various adjuvants are those conventionally used in the fields under consideration. Depending on their nature, these adjuvants can be introduced into the fatty phase, into the aqueous phase, into the lipid vesicles and/or into the nanoparticles and/or nanospheres.

When the cosmetic or dermatological composition of the invention is an emulsion, the proportion of the fatty phase can range from 5% to 80% by weight, and preferably from 5% to 50% by weight, relative to the total weight of the composition. The oils, the emulsifiers and the co-emulsifiers used in the composition in the form of an emulsion are chosen from those conventionally used in the field under consideration. The emulsifier and the co-emulsifier are present, in the composition, in a proportion ranging from 0.3% to 30% by weight, and preferably from 0.5% to 20% by weight, relative to the total weight of the composition.

As oils which can be used in combination with the oligonucleotides according to the invention, mention may be made of mineral oil (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax, ozokerite) can also be used as fatty substances.

As emulsifiers and co-emulsifiers which can be used in combination with the oligonucleotides according to the invention, mention may, for example, be made of esters of a fatty acid and of polyethylene glycol, such as PEG-20 stearate, and esters of a fatty acid and of glycerol, such as glyceryl stearate.

As hydrophilic gelling agents which can be used in combination with the oligonucleotides according to the invention, mention may in particular be made of carboxyvinyl polymers (carbomer), acrylic copolymers such as acrylate/alkyl acrylate copolymers, polyacrylamides, polysaccharides, natural gums and clays. As lipophilic gelling agents, mention may be made of modified clays such as bentones, metal salts of fatty acids, hydrophobic silica and polyethylenes.

A subject of the present invention is a cosmetic or dermatological composition containing at least one oligonucleotide described above and one or more other active agents.

The present invention also relates to a cosmetic or dermatological process for depigmenting and/or bleaching human skin, consisting in applying to the pigmented skin a cosmetic composition comprising an oligonucleotide sequence directed against the gene encoding tyrosinase. More particularly, the invention is directed towards a cosmetic treatment process for depigmenting or bleaching the skin, characterized in that it comprises the application of a composition defined above to one or more pigmented area(s) of the skin for a given period of time. Optionally, it is possible to repeat the operation, until the depigmenting effect has appeared. For this cosmetic application, the oligonucleotide content is preferably between 0.0003% and 3% of the total weight of the composition.

The present invention also relates to the use of an oligonucleotide as described above, for the manufacture of a medicament to be administered simultaneously, separately or sequentially in combination with one or more active agents.

The cosmetic composition of the invention can also comprise one or more active ingredients which can be used in cosmetics. Said active agents, in combination with the oligonucleotides according to the invention, are used pure or from extracts containing these active agents. They are in particular selected from the following compounds: ellagic acid and derivatives thereof; resorcinol and derivatives thereof; arbutin and derivatives thereof; vitamin C and derivatives thereof; pantothenate sulphonate and derivatives thereof; molecules which interfere directly or indirectly with alpha-melanocyte stimulating hormone (α-MSH) or its receptor or adrenocorticotropic hormone (ACTH); polyols such as glycerol, glycol or propylene glycol; keratolytic and/or desquamating agents such as salicylic acid and derivatives thereof; alpha-hydroxy acids such as lactic acid or malic agent, alone or grafted; retinoic acid; retinaldehyde; retinol and derivatives thereof such as the palmitate, propionate or acetate, in a preparation which may or may not be liposomal; antiglycation agents and/or antioxidants taken alone or in combination, such as tocopherol and derivatives thereof, ergothioneine, thiotaurine, hypotaurine, aminoguanidine, thiamine pyrophosphate, pyridoxamine, lysine, histidine, arginine, phenylalanine, pyridoxine, adenosine triphosphate; anti-inflammatory agents such as stearyl glcyrrhetinate; calmatives and mixtures thereof, one or more chemical or physical sunscreens such as octyl methoxycinnamate, butylmethoxydibenzoylmethane, titanium oxide and zinc oxide; and deoxyribonucleic and/or nucleic acids. In the event of incompatibility between these active agents and the oligonucleotides of the invention, one and/or the other can be incorporated into spherules, in particular liposomes or vesicles formed from amphiphilic, ionic or non-ionic lipids as described in French patent FR 2534487 and/or nanoparticles and/or nanospheres and/or nanocapsules.

The following examples illustrate the present invention without, however, limiting it.

In the Examples which follow, all the percentages are given by weight, unless otherwise indicated, and reference will be made to the figure legends.

### LEGENDS OF THE FIGURES RELATING TO THE RESULTS OF EXPERIMENTAL TRIALS

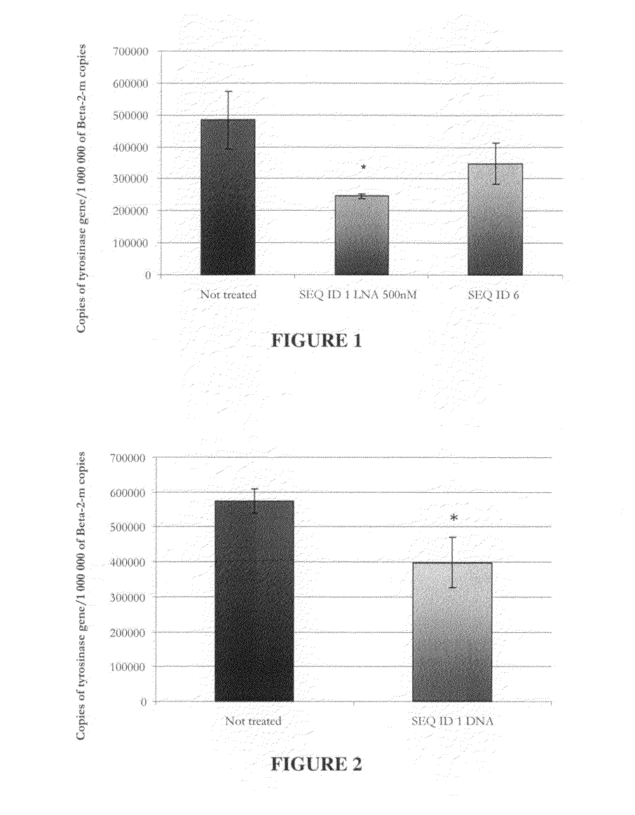

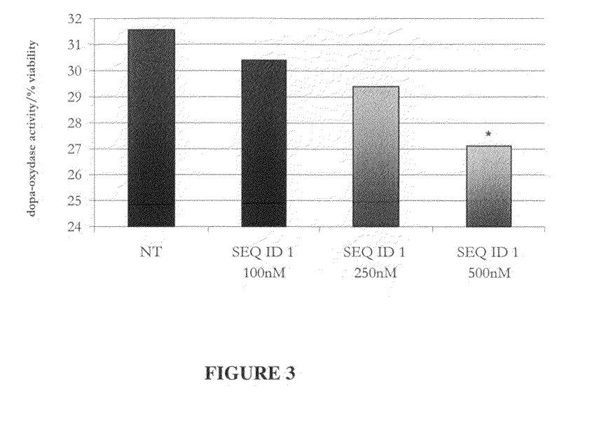

### EXAMPLE No. 3 for FIGS. 1 and 2, Example No. 4 for FIG. 3

FIG. 1: Effect of the oligonucleotide SEQ ID No. 1 composed of LNA bases and the oligonucleotide SEQ ID No. 6, on the expression of the tyrosinase gene in normal human melanocytes treated with 500 nM of oligonucleotides for 8 hours.

The symbol * represents a statistically significant difference (p<0.05) compared with the untreated cells.

FIG. 2: Effect of the oligonucleotide SEQ ID No. 1 composed of DNA bases, on the expression of the tyrosinase gene in normal human melanocytes treated with 500 nM of oligonucleotides for 8 hours.

The symbol * represents a statistically significant difference (p<0.05) compared with the untreated cells.

FIG. 3: Effect of the oligonucleotide SEQ ID No. 1 on the DOPA-oxidase activity of tyrosinase in normal human melanocytes.

The DOPA-oxidase activity is determined in OD per minute and standardized relative to the viability obtained using the XTT test. The symbol * represents a statistically significant difference (p<0.05) compared with the untreated cells.

### EXAMPLE 1

### Oligonucleotide Synthesis

The oligonucleotides with natural bases were synthesized with an automatic synthesizer (Perseptive Biosystems Expedite model 8909) using standard phosphoramidite derivative chemistry, using the constructor's protocols. The β-cyanoethyldiisopropyl-phosphoramidite derivatives of the nucleosides were used. The phosphite oxidation step was carried out with a solution of iodine. After cleavage from the column (Controlled Pore Class, Perseptive Biosystems) and complete deprotection of the sequence by treatment for 18 h at 55° C. with a 33% aqueous ammonia solution, the oligonucleotides were purified by precipitation from ethanol in the presence of sodium acetate. High pressure liquid chromatography controls were then carried out by ion exchange chromatography with elution using a gradient of sodium chloride, and by C18 reverse-phase chromatography with elution using a gradient of acetonitrile in the presence of triethylammonium acetate.

The LNA oligonucleotides were synthesized by the company Proligo or Eurogentec.

Oligonucleotides were synthesized by way of examples. They are described in Table 1. The stars placed next to the cytosines indicate that these bases are methylated in the 5-position. The first 5 sequences of this table, numbered from SEQ ID No. 1 to SEQ ID No. 5, were subjected to studies. Their property of hybridization with their target sequence on the tyrosinase gene and their depigmenting activity are reported in the following examples.

In Table 1, the numbers placed at each end of the sequences indicate the position of the oligonucleotide in the sequences of origin.

The sequences are deduced from the “HSTYRO1E” sequence of the human tyrosinase gene, published by Kikuchi et al., Biochem. Biophys. Acta 1009; 3; 283-286 (1989) (Genbank accession number M 27160) or from the “HSU03039” sequence of the human tyrosinase gene, published by Ponnazhagen et al., J. Invest. Dermatol., 102, 5, 744-748 (1994).

Moreover, by way of comparison so as to confirm the specificity of the oligonucleotides according to the invention with respect to the gene encoding tyrosinase, two oligonucleotides based on the sequence SEQ ID No. 1 of the invention were synthesized, namely:
an “inverted control” sequence, denoted SEQ ID No. 6 in Table 1, consisting in inverting the order of the bases of the sequence SEQ ID No. 1.
a “scrambled control” sequence, denoted SEQ ID No. 7, also in Table 1, comprising the same bases, in nature and in number, as those of the sequence SEQ ID No. 1, but placed in any order.

TABLE 1

| | | |
|---|---|---|
| SEQ | | |
| ID | | |
| No. | OLIGONUCLEOTIDE SEQUENCE | LOCUS |

| | | | | |
|---|---|---|---|---|
| 1. | 3535 | 5′-C*TTC*TC*TC*TTTTTC*C*TTTTTC*-3′ | 3555 | HSTYRO1E |
| 2. | 171 | 5′-TC*C*C*C*TC*TC*TC*TC*C*C*TC*TC*C*C*-3′ | 191 | HSTYRO1E |
| 3. | 201 | 5′-C*TC*TC*TC*TC*C*C*TC*TC*TC*TC*TC*-3′ | 221 | HSTYRO1E |
| 4. | 407 | 5′-C*TTTC*TTTC*TC*TC*TC*TC*TTTTC*T-3′ | 429 | HSU03039 |
| 5. | 1361 | 5′-TC*TC*TC*TC*C*TC*TTTC*C*TC*TC*T-3′ | 1381 | HSU03039 |
| 6. | | 5′-C*TTTTTC*C*TTTTTC*TC*TC*TTC*-3′ | | Inverted |
| | | | | SEQ ID |
| | | | | No. 1 |
| | | | | control |
| 7. | | 5′-TTC*TT C*TT C*TT C*TT C*TTC*TTC*-3′ | | Scrambled |
| | | | | SEQ ID |
| | | | | No. 1 |
| | | | | control |

C* denoting a 5-methylcytosine

### EXAMPLE 2

### Study of the Interaction of SEQ ID No. 1 with its Double Helix Target Sequence on the Human Tyrosinase Gene by Gel Shift

The gel shift technique makes it possible to demonstrate the hybridization of an oligonucleotide with its target DNA sequence. The bringing together of the target DNA with the radiolabelled oligonucleotide of the invention generates a DNA-oligonucleotide complex, the migration of which will be slowed down compared with that of the free oligonucleotide. The determination of the amount of oligonucleotide slowed down makes it possible to measure the effectiveness of formation of the triple helix.

Using solutions at 500 micromolar, SEQ ID No. 1 and SEQ ID No. 6 are labelled with γ-[³²P]ATP using the T4 polynucleotide kinase. The radiolabelled SEQ ID No. 1 and SEQ ID No. 6 are isolated on an exclusion column.

The purity of the oligonucleotides is verified by electrophoresis on a 15% acrylamide-urea gel, with migration at 70 W for 1 h 30. The gel is then dried under vacuum at 70° C. and the radioactive footprint is read using a PhosphoImager (Storm 860, Molecular Dynamics).

The hybridization between the labelled oligonucleotides and the DNA fragment comprising a portion of the sequence of the human tyrosinase gene is carried out overnight at 4° C. at various values for the target DNA/oligonucleotide concentration ratio. The conditions are mentioned in Table 2. The radiolabelled sequences SEQ ID No. 1 and SEQ ID No. 6 are tested in two forms: in a modified form (LNA) and in a non-modified form (DNA).

Electrophoresis on a non-denaturing 8% bisacrylamide gel is carried out for 1 h at a power of 3 W, at 37° C. The gel is then dried under vacuum at 70° C. and brought into contact with an exposure plate overnight.

TABLE 2
Gel shift reaction mixture

| | Concentration | | Volume in μl |
|---|---|---|---|
| Radiolabelled SEQ | 50 | nM | 0.5 |
| ID No. 1 | | | |
| Tyrosinase DNA | 50, 10 or 200 | nM | 3 |
| fragment | | | |
| Hepes | 50 | mM | 1.5 |
| MgCl₂ | 5 | mM | |
| NaCl | 150 | mM | |

From the image obtained, the effectiveness of formation of the complex between the oligonucleotide and its target sequence (expressed as a percentage) can be determined using the LumiAnalyst software (Roche, Meylan, France).

The results of the trials are reported in Table 3 below.

These results show that the yield from hybridization of SEQ ID No. 1, composed of LNA bases, with its target sequence varies according to the target DNA/oligonucleotide ratio. It is 11% for an equimolar ratio, 65% for a ratio of 2 and 77% for a ratio of 4. For SEQ ID No. 1, composed of DNA bases, the yield from hybridization with the tyrosinase gene fragment ranges from 25% to 33%, these values not being dependent on the target DNA concentration.

On the other hand, SEQ ID No. 6 does not interact with the tyrosinase DNA sequence, irrespective of whether the LNA derivatives or the DNA form are involved (Table 3).

TABLE 3
Quantification of the hybridization of SEQ ID
No. 1 with the tyrosinase gene fragment, using the
LumiAnalyst software

| | 50 mM | 100 mM | 200 mM |
|---|---|---|---|
| | tyrosinase | tyrosinase | tyrosinase |
| | gene DNA | gene DNA | gene DNA |
| % bound SEQ ID No. | 11 | 65 | 77 |
| 1 (LNA derivatives) | | | |
| % free SEQ ID No. 1 | 89 | 35 | 23 |
| (LNA derivatives) | | | |
| % bound SEQ ID No. | 26 | 25 | 33 |
| 1 (DNA) | | | |
| % free SEQ ID No. 1 | 74 | 75 | 67 |
| (DNA) | | | |
| % bound SEQ ID No. | 0 | 0 | 0 |
| 6 (LNA derivatives) | | | |
| % free SEQ ID No. 6 | 100 | 100 | 100 |
| (LNA derivatives) | | | |
| % bound SEQ ID No. | 0 | 0 | 0 |
| 6 (DNA) | | | |
| % free SEQ ID No. 6 | 100 | 100 | 100 |
| (DNA) | | | |

### EXAMPLE 3

### Study by Real-Time Quantitative RT-PCR of the Inhibition of Human Tyrosinase Gene Expression on Normal Human Melanocytes

Normal human melanocytes are seeded into Petri dishes 60 mm in diameter, at a rate of 710 000 cells per dish, in medium 1 (Table 4). For each of the sequences SEQ ID Nos. 1 to 7, an extemporaneous solution is prepared consisting of medium 1 to which has been added the sequence complexed with Superfect (Qiagen, Courtabœuf, France), at the concentration of 500 nanomolar. Twenty-four hours after the seeding, the cells are treated once with these various solutions, and then recovered after 8 hours of treatment in order to extract the total RNA therefrom.

TABLE 4
Composition of medium 1

| | | | Final | |
|---|---|---|---|---|
| | | | concentration per | |
| | Reference | Supplier | ml of KSFM medium | |
| KSFM | 17005-075 | Invitrogen | | |
| Pituitary | 17005-075 | Invitrogen | 50 | μg/ml |
| extracts | | | | |
| EGF | 17005-075 | Invitrogen | 5 | ng/ml |
| β-FGF | 13256-029 | Invitrogen | 10 | ng/ml |

The total RNA (all the cellular RNA, therefore comprising the tyrosinase messenger RNA) is extracted (Rneasy Kit, Qiagen, Courtabœuf, France), and assayed, and its quality verified using the bioanalyser 2100 (Agilent Technologies, Massy, France). It is then treated with DNAase, in order to eliminate all traces of possibly residual genomic DNA. In order to carry out the real-time quantitative PCR reaction, which is a DNA amplification reaction, a reverse transcription reaction with the Superscript II enzyme (Qiagen) makes it possible to generate the complementary DNAs (cDNAs) from the RNAs. Using this reverse transcription product which has been obtained, the real-time quantitative PCR is then carried out on a LightCycler (Roche, Meylan, France) according to the reaction mixture described in Table 5 in order to quantify the messenger RNA of interest present in the initial cell population.

TABLE 5
Standard reaction mixture used for the real-
time quantitative PCR

| | Final | Volume in |
|---|---|---|
| | concentration | μl |
| SYBRGreen mix | dilution 1/10 | 2 |
| (dNTP, Taq, pol) | | |
| Sense primer | 0.5 μM | 2 |
| Antisense primer | 0.5 μM | 2 |
| MgCl₂ | 3.5 mM | 2 |
| H₂O | / | 10 |

2 μl of the product from the reverse transcription reaction described above and containing the cellular cDNAs are added to the standard reaction mixture used for the real-time quantitative PCR reaction and described in Table 5. The amount of transcripts encoding the tyrosinase gene measured for a sample is related to the amount of transcripts encoding the non-varying beta-2-microglobulin gene. The results obtained for this ratio are recapitulated in Table 6 below, and represented in the attached FIGS. 1 and 2. SEQ ID No. 1 composed of LNA bases decreases, by 49%, the amount of tyrosinase gene transcripts in a significant manner; for SEQ ID No. 1 composed of DNA bases, the decrease in the amount of transcripts is 30%. On the other hand, the sequences SEQ ID Nos. 2 to 5 do not exhibit any significant activity on the inhibition of tyrosinase gene expression, nor even do the sequences SEQ ID No. 6 and SEQ ID No. 7, which are, respectively, the inverted control and the scrambled control for the sequence SEQ ID No. 1, which is the only one to possess this activity.

TABLE 6
Recapitulation of the effect of the sequences
SEQ ID No. 1 to SEQ ID No. 7 on the amount of
transcripts encoding the tyrosinase gene

| Treatments | % inhibition |
|---|---|
| SEQ ID No. 1 LNA bases | 49 |
| SEQ ID No. 1 DNA bases | 30 |
| SEQ ID No. 2 | NS |
| SEQ ID No. 3 | NS |
| SEQ ID No. 4 | NS |
| SEQ ID No. 5 | NS |
| SEQ ID No. 6 | NS |
| SEQ ID No. 7 | NS |

### EXAMPLE 4

### Study of the Inhibition of Human Tyrosinase Gene Expression on Normal Human Melanocytes, by Measuring the Dopa-Oxidase Activity of Tyrosinase

The oligonucleotides according to the invention which appear in Table 1 of Example 1 (SEQ ID No. 1 to SEQ ID No. 5) are studied with respect to their action on melanogenesis, and therefore with respect to their ability to modulate the production of melanin pigments by melanocytes. As elements of comparison, the “inverted control” (SEQ ID No. 6) and “scrambled control” (SEQ ID No. 7) oligonucleotides are also studied with respect to this action.

It is recalled that, during the reaction mechanism for melanogenesis, tyrosinase and TRP-1 form an enzymatic complex which catalyses the conversion of L-dopa to dopaquinone and then to dopachrome (Kobayashi T. et al., J. Biol. Chem. 273, 31810-5 (1998); Sarangarajan R., Boissy R. E., Pigment Cell Res., 16, 437-44 (2001)). It should be noted that tyrosinase is also called “dopa-oxidase” due to its multifunctional nature. It in fact performs the same function as dopa-oxidase during the oxidation of tyrosine to dopa.

The principle of the present test is based on measurement of the rate of reaction catalysed by dopa-oxidase in the conversion of L-dopa, used as substrate, to dopachrome. The appearance of dopachrome is quantified by measuring optical density at given time periods, which makes it possible to calculate the reaction rate for L-dopa conversion for each of the oligonucleotides tested and for the control assay. Given that the rate of this reaction depends in particular on the amount of enzyme available, it will be possible, from the results obtained, to verify the inhibitory action of the oligonucleotides of the invention which are tested, on tyrosinase gene expression, which action was already observed by means of the tests of the previous example. A decrease in the reaction rate compared to the control assay will correspond to a decrease in the amount of enzyme formed, and therefore a decrease in the enzymatic activity and, consequently, to reduced melanin formation and therefore to a depigmenting effect.

Normal human melanocytes are seeded into a 96-well microplate at a rate of 10 000 cells per well in medium 1 (Table 4). For each of the sequences SEQ ID Nos. 1 to 7, an extemporaneous solution is prepared consisting of medium 1 to which has been added the sequence complexed with Superfect (Qiagen, Courtabœuf, France), at the concentration of 500 nanomolar. Twenty-four hours after seeding, the cells are treated once, and then the dopa-oxidase activity is measured after 72 hours of treatment, by spectrophotometry at 450 nm. The results are given in Table 7 and represented in the attached FIG. 3.

TABLE 7
Recapitulation of the effect of the sequences
SEQ ID No. 1 to SEQ ID No. 7 on the dopa-oxidase
activity of the tyrosinase of normal human melanocytes

| | % decrease in dopa-oxidase |
|---|---|
| Treatments | activity |
| SEQ ID No. 1 (LNA | 14 |
| bases) | |
| SEQ ID No. 2 | NS |
| SEQ ID No. 3 | NS |
| SEQ ID No. 4 | NS |
| SEQ ID No. 5 | NS |
| SEQ ID No. 6 | NS |
| SEQ ID No. 7 | NS |

Table 7 shows that the DOPA-oxidase activity is significantly inhibited, by 14%, in the normal human melanocytes treated with SEQ ID No. 1 at 500 nanomolar. SEQ ID No. 6 and SEQ ID No. 7 have no activity on the DOPA-oxidase activity. This shows that the inhibitory effect observed with sequence SEQ ID No. 1 composed of LNA bases is specific for the targeted sequence.

### EXAMPLE 5

### Cosmetic Powder for Lightening the Facial Complexion

| | |
|---|---|
| Microcellulose | 20.00% |
| Sodium lauryl sulphoacetate | 15.00% |
| Oligonucleotide SEQ ID No. 1 | 1.00% |
| Fragrance, dyes, preserving agents | qs |
| Talc | qs 100% |

This powder has a double action. It allows cleansing of the skin, and it also makes it possible, through regular use for a few days, to lighten the complexion. It can be applied to the facial skin once or twice a day.

### EXAMPLE 6

### Depigmenting Cosmetic Day Cream in Gel-Emulsion Form

| | |
|---|---|
| Glycerol | 5.00% |
| Caprylic/capric/succinic triglycerides | 5.00% |
| Octyl methoxycinnamate | 1.00% |
| Dimethicone copolyol | 0.50% |
| Acrylates/C₁₀₋₃₀ alkyl acrylate crosspolymer | 0.50% |
| Oligonucleotides SEQ ID No. 1 (LNA) | 0.01% |
| Neutralizing agent | qs |
| Preserving agents, fragrance, dyes, | qs |
| Water | qs 100% |

Some individuals subjected to the more or less intense radiation of daylight, or even of the sun directly, wish to conserve a light complexion and to avoid the appearance of pigmented spots. The use of the gel-emulsion above will make it possible to achieve this aim. This composition is applied to the face, generally in the morning. It acts both preventively and curatively on even or uneven pigmentation of the face.

### EXAMPLE 7

### Fluid Cosmetic Composition which Protects Against Radiation from the Sun (SPF 30)

| | |
|---|---|
| Volatile pentacyclomethicone | 49.00% |
| Titanium dioxide | 15.00% |
| Octyl methoxycinnamate | 7.50% |
| Glycerol | 5.00% |
| Phenyl trimethicone | 5.00% |
| Dimethicone copolyol | 3.00% |
| Poly (methyl methacrylate) | 2.50% |
| Butylmethoxydibenzoylmethane | 1.00% |
| Oligonucleotide SEQ ID No. 1 | 0.1% |
| Neutralizing agent, fragrance, preserving | qs |
| agents, antioxidants | |
| Water | qs 100% |

This composition is to be used before exposure to strong radiation from the sun. It prevents the appearance of pigmentary spots in individuals predisposed to this phenomenon. It should be noted that the presence of a high concentration of sunscreen makes it possible to compensate for the decrease in natural protection which results from the decrease in the amount of melanin.

### EXAMPLE 8

### Dermatological Cream for the Treatment of Skin Hyperpigmentations of Pathological or Trauma-Based Origin

| | |
|---|---|
| Glyceryl stearate + PEG-100 stearate | 5.00% |
| Hydrogenated polyisobutene | 4.00% |
| Magnesium ascorbyl phosphate | 3.00% |
| Glyceryl tricaprylate/caprate | 3.00% |
| Squalane | 3.00% |
| Glycerol | 2.00% |
| Beeswax | 1.50% |
| Cetearyl octanoate | 1.50% |
| Cetyl alcohol | 1.00% |
| Stearyl alcohol | 1.00% |
| Dimethicone | 1.00% |
| Xanthan gum | 0.30% |
| Ethylenediaminetetraacetic acid | 0.20% |
| Citric acid | 0.10% |
| Sodium citrate | 0.10% |
| Oligonucleotide SEQ ID No. 1 | 0.10% |
| Neutralizing agent, fragrance, preserving | qs |
| agents | |
| Water | qs 100% |

The use of this cream makes it possible to reduce skin hyperpigmentations of pathological or trauma-based origin. This cream also makes it possible to reduce the colour contrast at the periphery of depigmented areas in the case of vitiligo.

### EXAMPLE 9

### Cosmetic Face Lotion for Lightening the Complexion

| | |
|---|---|
| Ethyl alcohol | 30.00% |
| PPG-3 myristyl ether | 5.00% |
| Glycerol | 2.00% |
| Carbomer | 0.20% |
| Polysorbate 20 | 0.20% |
| Oligonucleotide SEQ ID No. 1 (LNA) | 0.01% |
| Neutralizing agent, fragrance, preserving | Qs |
| agents | |
| Water | qs 100% |

This lotion for lightening the complexion is used after removing make-up from the skin and cleansing the latter.

### EXAMPLE 10

### Cosmetic Facial Lightening Serum

| | |
|---|---|
| Water | qs 100% |
| Glycerol | 2% |
| Tetrasodium EDTA | qs desired |
| Citric acid | |
| Trisodium citrate | pH |
| Xanthan gum | 0.25% |
| Polyacrylamide, C13.14 isoparaffin, | 0.5% |
| laureth-7 | |
| Dimethicone copolyol | 0.25% |
| Oligonucleotide SEQ ID No. 1 | 0.1% |
| Fragrance, dye, preserving agent | qs |

A drop of this very concentrated serum composition is applied to the face, generally before the application of a face cream. This serum is normally used as treatments of one to two weeks so as to obtain or maintain a lightening of the complexion.

### EXAMPLE 11

### Cosmetic Lotion for Lightening Body Hair

| | |
|---|---|
| Water | qs 100% |
| Alcohol | 50% |
| Panthenyl ethyl ether | 0.5% |
| DL-α-tocopheryl acetate | 0.2% |
| Polysorbate 60 | 1% |
| Oligonucleotide SEQ ID No. 1 | 0.01% |
| Fragrance | 0.2% |
| Glycerol | 0.5% |
| Dye | qs |

This lotion is applied to the areas with hair that are to be lightened, in particular the arms, for the amount of time sufficient to obtain gradual lightening of the hairs.

### EXAMPLE 12

### Cosmetic Anti-Spot Gel-Cream for the Hands

| | |
|---|---|
| Caprylic/capric diglyceryl succinate | 6% |
| Octyl octanoate | 2.5% |
| Octyl methoxycinnamate | 6% |
| Oligonucleotide SEQ ID No. 1 | 0.001% |
| Phenyltrimethicone | 2.5% |
| Benzophenone-3 | 0.5% |
| Sodium hyaluronate | 0.05% |
| Xanthan gum | 0.2% |
| Acrylates/C10.30 alkyl acrylate copolymer | 0.5% |
| Glycerol | 2% |
| PEG 150 | 3% |
| Neutralizing agents, dyes, fragrance, | Qs |
| preserving agents | |
| Purified water | qs 100% |

This cream must be applied directly to the spots (solar and/or senile lentigo) on the hands, so as to reduce the colouration of said spots.

1. An anti-gene oligonucleotide comprising a sequence of 18 to 21 nucleotides comprising the sequence 5′-C*TTC*TC*TC*TTTTTC*C*TTTTTC*-3′ (SEQ ID No. 1, C* denoting a 5-methylcytosine) hybridising specifically with the gene encoding for human tyrosinase by Hoogsteen pairing between the complementary bases, said oligonucleotide forming a triple-helix structure with the human tyrosinase gene.

2. The oligonucleotide according to claim 1 comprising 21 nucleotides.

3. The oligonucleotide according to claim 1, characterized in that it is a chimeric oligonucleotide comprising at least one chemical modification at the level of the sugar part, the nucleobase part and/or the internucleotide skeleton.

4. The oligonucleotide according to claim 3, characterized in that it is a LNA (Locked Nucleic Acid) or a PNA (Peptide Nucleic Acid).

5. The oligonucleotide according to claim 3, characterized in that it comprises at least one modification selected from the group of 2′-O-alkyl and 2′-O-fluoro derivatives on the sugar part of the nucleoside, phosphorothioates or methylphosphonates derivatives at the level of the internucleotide skeleton, carrier derivatives in 5′ and/or 3′ of an—intercalating reactive group the type, in particular acridine, and derivatives for establishing cross-linking with the target sequence, in particular psoralene and an azide group.

6. A cosmetic or dermatological composition, characterized in that it comprises at least an oligonucleotide according to claim 1.

7. Composition according to claim 6, characterized in that the oligonucleotide is present in an amount ranging from 0.00001% to 10%, and preferably from 0.0003% to 3%, of the total weight of the composition.

8. Composition according to claim 6, characterized in that it is in a form suitable for topical application, in particular in the form of an aqueous, aqueous-alcoholic or oily solution, a simple or multiple, oil-in-water or water-in-oil emulsion, an aqueous or oily gel, a liquid, pasty or solid anhydrous product, an aqueous or oily dispersion of solid particles, such as polymeric nanospheres or nanocapsules, or else an aqueous dispersion of lipid vesicles of ionic or non-ionic type.

9. Cosmetic composition according to claim 6, characterized in that it is formulated so as to be used as a cosmetic care product and/or as a make-up product.

10. Composition according to claim 6, characterized in that it also comprises one or more active ingredients which can be used in cosmetics, in particular one or more chemical or physical sunscreens.

11. Cosmetic treatment process for depigmenting or bleaching the skin, characterized in that it comprises the application of a composition defined according to claim 6 to one or more pigmented area(s) of the skin for a given period of time, and optionally repeating the operation, until the depigmenting effect has appeared.

12. Cosmetic treatment process for depigmenting or bleaching body hair, characterized in that it comprises the application of a composition defined according to claim 6 to one or more area(s) of the skin with hair for a determined period of time, and optionally repeating the operation, until the depigmenting effect has appeared.

13. Cosmetic treatment process according to claim 11, characterized in that the oligonucleotide content is between 0.0003% and 3% of the total weight of the composition.

14. Use of an oligonucleotide according to claim 1, for the manufacture of a medicament to be applied topically, for use in the treatment or prevention of diseases reflected by the overexpression and the overactivity of tyrosinase, in particular for the treatment or prevention of regional hyperpigmentations due to melanocyte hyperactivity, such as idiopathic melasmas, localized hyperpigmentations due to benign melanocyte proliferation and hyperactivity, such as pigmentary senescence spots (senile lentigo), and accidental hyperpigmentations such as photosensitization or post-lesional cicatrization, and for the treatment of forms of leucodermia such as vitiligo.
